(19) United States
(12) Patent Application Publication
(10) Pub. No.: US 2025/0352603 A1
(43) Pub. Date: Nov. 20, 2025

(54) VALERIAN COMPOSITION FOR SLEEP MANAGEMENT

(71) Applicant: OMNIACTIVE HEALTH TECHNOLOGIES LIMITED, Mumbai, Maharashtra (IN)

(72) Inventors: Prakash BHANUSE, Thane (W), Maharashtra (IN); Gaurav JHA, Thane (W), Maharashtra (IN); Rohini VERMA, Thane (W), Maharashtra (IN); Keshav DAREKAR, Thane (W), Maharashtra (IN)

(73) Assignee: OMNIACTIVE HEALTH TECHNOLOGIES LIMITED, Mumbai, Maharashtra (IN)

(21) Appl. No.: 18/570,959

(22) PCT Filed: Jan. 17, 2023
(86) PCT No.: PCT/IB2023/050410

§ 371 Date: Dec. 15, 2023

(30) Foreign Application Priority Data
Jan. 18, 2022 (IN) 202221002782

(51) Int. Cl. A61K 36/84; A61K 9/16; A61P 25/20

(52) U.S. Cl. CPC A61K 36/84; A61K 9/1611; A61K 9/1617; A61P 25/20

(57) ABSTRACT

The present invention is related to the valerian composition and/or valerian extract for prevention, improvement, and maintenance of sleep disorders and/or sleep management per se improve the quality sleep, improves time to sleep, improves sleep duration, reduces sleep disturbances, maintain healthy stress levels to a subject in need thereof. A valerian composition comprising of the valerian extract and at least one pharmaceutically and/or nutraceutically accepted excipients. The present invention further encompasses the valerian composition wherein further formulated in different forms like powder, beadlets, granules, capsules, tablets, oil suspension, films or any other suitable oral or topical dosage forms using pharmaceutically and/or nutraceutically acceptable excipients.

### RELATED APPLICATIONS

This application is a U.S. National Stage application of International Patent Application Number PCT/IB2023/050410, filed on Jan. 17, 2023, which claims the benefit of priority of the Indian provisional application number IN 202221002782, filed on Jan. 18, 2022, all of which are incorporated herein by reference in their entirety.

### FIELD OF THE INVENTION

The present invention is related to a valerian composition and/or valerian extract for the prevention and/or improvement of sleep disorders in a subject in need thereof. More particularly, the invention describes a valerian composition comprising valerian extract and at least one pharmaceutically and/or nutraceutically acceptable excipient. The invention is further related to a composition wherein the valerian extract comprises valerenic acids, hydroxy valerenic acid, and acetoxy valerenic acid for the prevention and/or improvement of sleep-related disorders.

Also, the invention is related to the process of extraction and isolation of valerian extract from the valerian plant. In addition, the invention is related to the process of preparation of a valerian composition comprising valerian extract and at least one pharmaceutically and/or nutraceutically accepted excipients. Also disclosed herein is a valerian composition formulated in different forms such as powder, beadlets, granules, capsules, tablets, oil suspension, films, or any other suitable oral or topical dosage forms using pharmaceutically and/or nutraceutically acceptable excipients.

### BACKGROUND OF THE INVENTION

Insomnia is one of the most common sleep disorders among adults. Numerous surveys conducted in countries around the world report that approximately 30% to 40% of adults have problems initiating or maintaining sleep. A smaller percentage of adults report severe problems (10%-15%), but the prevalence of severe, chronic sleep problems increases to 25% in the elderly. Insomnia is also more common in patients with chronic medical problems and is found in up to 69% of patients enrolled in primary care clinics.

Approximately 40% of adults with insomnia have used either over-the-counter medication or alcohol to help induce sleep, and approximately one-quarter have used prescription medications at least once. There is only limited evidence to support the efficacy of many of the commonly used medications for insomnia, including antihistamines, chloral hydrate, barbiturates, tryptophan, and melatonin. Although benzodiazepines are known to be effective for insomnia, the clinical benefit is small (<1 hour of increased sleep) and similar to that found with exercise therapy alone. Moreover, chronic benzodiazepine therapy for sleep is associated with several negative side effects, including cognitive impairment and an increased risk of motor vehicle accidents, falls, and fractures.

The extract of the root of valerian (Valeriana officinalis), a flowering plant, native to temperate regions of the Americas, Europe, and Asia. Active compounds present in valerian includes flavonones, alkaloids and sesquiterpenes. These extracts contain a number of chemicals with CNS activity, including sesquiterpenes, valepotriates, valerenic acid, and various other alkaloids, in unknown proportions. The constituents of a specific valerian preparation also depend on the actual valerian species used, the method of extraction.

U.S. Pat. No. 6,383,526 B1 relates to a process for isolating a pharmaceutically active extract from a plant root. More specifically, the present invention relates to a process for maximizing the yield of valerenic acid and other valerenic acids and their derivatives, while simultaneously minimizing the yield of valepotriates and valepotriates decomposition products or derivatives in an extract of the root of the plant Valeriana officinalis L.

US 2004/0241255 A1 relates to pharmaceutical compositions and methods for treating human patients suffering from sleep-induced apnea or hypopnea. Patients suffering from hypopnea (too shallow breathing) or apnea (more or less periodical interruptions of breathing) while sleeping tend to develop sleep deficiencies when awake that may lead to reduced vigilance and increasing sleepiness.

Thus, there is a need of effective and convenient solutions for sleep related disorders. Considering their high prevalence, precise methodological clinical trials of natural sleep aids such as valerian are needed. The use of valerian extract can be an important treatment alternative because it is a naturally available ingredient with relatively inexpensive and without side effects. For the successful industrialization of these products, extraction optimization, preparation of active, compound-rich products, and standardization should be further investigated. Therefore, it is important to understand the sleep-promoting effects of valerian composition and/or valerian extract with the help of a suitable animal model.

### BRIEF DESCRIPTION OF THE DRAWINGS

FIG. 1 shows the effect of melatonin and valerian extracts (VA) on brain electrical activity. Spike frequency and amplitude analyses on ECoG recording. Values are represented as mean±S.E.M. for each group, n=5. A) Spike amplitude (%) and B) Spike frequency (%).

FIG. 2 shows the effects of melatonin and valerian extracts (VA) on the sleep duration (A) and sleep latency (B) after pentobarbital administration. Each bar represents the mean and standard error of the mean. Tukey's post-hoc test were used for comparing the results among different treatment groups, and statistical significance between groups is shown by: ###P<0.001; P<0.0001 compared to P group, *P<0.05 compared to PM group, and &&& P<0.001; && P<0.0001 compared to PVA I group.

FIG. 3 shows the effects of melatonin and valerian extracts (VA) on the serum of serotonin, dopamine, and melatonin. ANOVA and Tukey's post-hoc test were used for comparing the results among different treatment groups, and statistical significance between groups is shown by: ** P<0.01; *** P<0.001, **** P<0.0001 compared to control group, ###P<0.001; ####P<0.0001 compared to C group, ++++P<0.0001 compared to CM group.

FIG. 4 shows the effects of melatonin and valerian extracts (VA) on the brain tissue MDA (A), SOD (B), CAT (C), and GPx. ANOVA and Tukey's post-hoc test were used for comparing the results among different treatment groups, and statistical significance between groups is shown by: *** P<0.001, **** P<0.0001 compared to control group, ##P<0.01; ###P<0.001; ####P<0.0001 compared to C group, ++P<0.01 compared to CM group, & P<0.05 compared to CVA I group.

FIG. 5 shows the effects of melatonin and valerian extracts (VA) on the brain tissue GABAergic receptors GABA_{A}R2 (A), GABA_{B} R1 (B), GABA_{B} R2 (C), and serotonergic receptor 5-HT1A (D). ANOVA and Tukey's post-hoc test were used for comparing the results among different treatment groups, and statistical significance between groups is shown by: **** P<0.0001 compared to control group, #P<0.05; ##P<0.01; ###P<0.001; ####P<0.0001 compared to C group, * P<0.05; +++P<0.001; ++++P<0.0001 compared to CM group, and && P<0.01; &&&&P<0.0001 compared to CVA I group.

FIG. 6 shows the effects of melatonin and valerian extracts (VA) on the brain GluA1 (A), GluN2A (B), and GluN1 (C). ANOVA and Tukey's post-hoc test were used for comparing the results among different treatment groups, and statistical significance between groups is shown by: ** P<0.01; **** P<0.0001 compared to control group, ####P<0.0001 compared to C group, +P<0.05 compared to CM group, and &&&& P<0.0001 compared to CVA I group.

FIG. 7 shows the effects of melatonin and valerian extracts (VA) on the brain Bax(A), Bcl-2 (B), and Caspase-3 (C). ANOVA and Tukey's post-hoc test were used for comparing the results among different treatment groups, and statistical significance between groups is shown by: **** P<0.0001 compared to control group, ###P<0.001; ####P<0.0001 compared to C group, +P<0.05; ++++P<0.0001 compared to CM group, and &&&& P<0.0001 compared to CVA I group.

### OBJECTIVES OF THE INVENTION

The primary objective of the present invention is to provide the valerian composition comprising valerian extract.

The further objective of the present invention is to provide valerian composition and/or valerian extract comprising valerenic acid, hydroxy valerenic acid, and acetoxy valerenic acid in an effective ratio.

Another objective of the present invention is to provide the valerian composition comprised valerian extract as per the objectives use for prevention and/or improvement of sleep disorders to a subject in need thereof.

Further objective of the present invention is to provide the valerian composition comprising valerian extract as per the objectives for improving quality of sleep, improving time to sleep, improving sleep duration, reducing sleep disturbances, and maintaining healthy stress levels.

Another objective of the invention is to provide a process for extraction comprising of a valerian extract constituent from valerian plant.

Further objective of the present invention is to provide a process for the preparation of the valerian composition comprising one or more pharmaceutically and/or nutraceutically accepted excipients.

Further objective of the present invention is to provide a valerian composition with at least one pharmaceutically and/or nutraceutically accepted excipient, wherein the composition is formulated in different forms, for example, orally administrable solid, semisolid, liquid forms, selected from, but not limited to dosages such as, powders, granules, pellets, beadlets, caplets, tablets, capsules, soft gel capsules, solution, emulsions, suspensions, oil suspensions, dispersions, and the likes.

### DETAILED DESCRIPTION OF THE INVENTION

Valerian (Valeriana officinalis) is a medicinal plant endogenous to Europe, Asia and widely introduced in North America. Various portions of the plant are consumed for medicinal reasons, including the flowers, fruit, and leaves. However, it is valerian root that is considered to be most psychoactive, and its extracts are used as hypnotics and anxiolytics. Active compounds present in valerian includes flavonones, alkaloids and sesquiterpenes. These extracts contain a number of chemicals with CNS activity, including sesquiterpenes, valepotriates, valerenic acid, and various other alkaloids, in unknown proportions. Valerenic acid is a monocarboxylic acid that is 2-methylprop-2-enoic acid which is substituted at position 3 by a 3,7-dimethyl-2,4,5,6,7,7a-hexahydro-1H-inden-4-yl group. A bicyclic sesquiterpenoid constituent of the essential oil of the Valerian plant.

The present invention is to mainly provide the valerian compositions comprises valerian extract.

Also the present invention described herein relates to a valerian composition comprises valerian extract use for prevention and improvement of sleep disorders.

It is to be appreciated that the term “valerian extract” can be interpreted to be within the scope of, which comprises valerenic acid either alone or in combination with hydroxy valerenic acid and/or acetoxy valerenic acid. It is also appreciated that the total valerenic acid including hydroxy valerenic acid and acetoxy valerenic acid is in the range of 5-20% of the extract.

The total valerenic acid present in the extract is in the range of 7-12% of the extract.

The total valerenic acid present in the extract is in the range of 7.5-10% of the extract.

According to embodiment of the present invention “valerian compositions” of the invention are comprised of the valerian extract as defined above and one or more of pharmaceutically and/or nutraceutically accepted excipients.

In some of the embodiments of the present invention there is provided a process for preparing valerian extract of the invention from plant parts of “Valeriana officinalis” that comprises:
a) obtaining plant material from one or more parts of the valerian plants,
b) obtaining an extract from the plant material by soaking it into suitable aqueous or an organic solvent, or a combination thereof for a suitable time period,
c) stirring the soaked plant material for a defined period of time, and
d) adding suitable aqueous or an organic solvent, or a combination thereof, allow to settle at room temperature and filter/decant to remove the extract material.

According to further embodiment, suitable aqueous solvent can be selected from water, and organic solvent can be selected from methanol, ethanol, propanol, acetone, ethyl acetate chloroform alone or in combination thereof.

According to further embodiment the valerian extract comprises total valerenic acid not less than 8% w/w of total extract.

According to further embodiment the valerian extract comprises total valerenic acid not less than 5% w/w of total extract.

According to further embodiment the valerian extract comprises hydroxy valerenic acid not less than 0.1% w/w of total extract.

According to further embodiment the valerian extract comprises acetoxy valerenic acid not less than 1.0% w/w of total extract.

According to further embodiment the valerian extract comprises hydroxy valerenic acid not less than 3% w/w of total extract.

More preferably, valerian compositions are formulated using excipients selected from the group of, but not limited to solvents, anticaking agent, bioavailability enhancer, solublizer, emulsifier, binder, filler, pH modifier and coating agent or the combination thereof.

According to further embodiment compositions described herein can be available in orally administrable solid, semisolid, liquid forms, selected from, but not limited to dosages such as, powders, granules, pellets, beadlets, caplets, tablets, capsules, soft gel capsules, solution, emulsions, suspensions, oil suspensions, dispersions and the like.

The valerian compositions, as described herein are comprised of valerian extract and at least one excipient such as pharmaceutically and/or nutraceutically accepted excipients which can be formulated as granules, powder and/or beadlet.

In some embodiments, a process for the preparation of the valerian composition is in the form of powder comprises:
(i) Added suitable solvent and valerian extract in clean vessel under stirring to obtain clear solution,
(ii) Added suitable solvent and carrier under stirring to obtain clear solution in another vessel,
(iii) Added solution obtain in step (i) under stirring into step (ii) solution under stirring for a required period,
(iv) Spray dry solution obtain from step (iii), and
(v) Add anti-caking agent in spray dry powder obtained in step (iv) to obtain valerian composition in powder form.

In one more embodiment, the suitable solvent is used in preparation of a valerian composition is selected from the group such as, but not limited to water, methanol, ethanol, Iso propyl alcohol, acetone, ethyl acetate, methylene dichloride and/or combination thereof.

In one more embodiment, the carrier is used in preparation of a valerian composition is selected from the group such as, but not limited to hydroxypropyl methyl cellulose and cellulose derivatives, microcrystalline cellulose, polyacrylates, polyethylene glycols, povidones, starch and starch derivatives, chitosan, gums, polyvinyl alcohol, dextran, polylactic-co-glycolic acid, polyethylene glycol, maltodextrin and/or combination thereof.

In one more embodiment, the anticaking agent in preparation of a valerian composition is selected from, but not limited to Silicon dioxide, dicalcium phosphate, magnesium stearate, stearic acid, mannitol and/or combination thereof.

In some embodiments, a process for the preparation of the valerian composition in form of beadlet comprises:
(i) adding solvent in clean vessel along with valerian extract, bioenhancer, solubilizer and emulsifier under stirring to obtain clear solution,
(ii) adding binder and solvent in another vessel,
(iii) adding filler, pH modifier and anticaking agent under stirring to form uniform powder blend,
(iv) adding solution prepared in step (i) in the dry blend of step (ii) to obtain the wet mass, (v) adding solution prepared in step (iii) in wet mass obtained in step (iv),
(vi) passing the wet mass through extruder followed by spheronizer,
(vii) drying and sifting the granules obtain in step (vi),
(viii) preparing a coating solution using coating agent and solvent,
(ix) loading granules obtained in step (vii) in fluidized bed coater along with solution obtained in step (viii), and
(x) drying the obtained granules in step ix.

In one more embodiment, the bioavailability enhancer employed in preparation of a valerian compositions is selected from the group such as, but not limited to, d-limonene, monoglycerides, and phospholipids such as lecithin/phosphatidyl choline, Vegetable Oils, and botanical extracts combination thereof.

In one more embodiment, the solubilizer in preparation of a valerian composition is selected from the group such as, but not limited to MCT oil, polysorbate, propylene glycol alginate, sugar alcohols, sugar esters, phospholipid, Vitamin E TPGS (d-α-Tocopheryl polyethylene glycol 1000 succinate), beta cyclodextrin and/or combination thereof.

In one more embodiment, the emulsifier in preparation of a valerian composition is selected from the group such as, but not limited to, steric acid, phosphatidylcholine, polysorbate, sugar alcohols, glycerol & derivatives and/or combination thereof.

In one more embodiment, the binder in preparation of a valerian composition is selected from the group such as, but not limited to hydroxy propyl cellulose, pregelatinized starch and/or combination thereof.

In one more embodiment, the filler in preparation of a valerian composition is selected from the group such as, but not limited to microcrystalline Cellulose, Maize Starch, Lactose, Maltose and/or combination thereof.

In one more embodiment, the pH modifier in preparation of a valerian composition is selected from the group such as, but not limited to sodium bicarbonate, citric acid, trisodium citrate, lactic acid, L-arginine, calcium carbonate, magnesium carbonate, and/or combination thereof.

In one more embodiment, the coating agent in preparation of a valerian composition is selected from the group such as, but not limited to stearic acid, glyceryl di-stearate, shellac, zein, acrylate polymer, cellulose polymer, and/or combination thereof.

### Powder Formulation Preparation

### Example 1

| Sr. | | Functional | % | Qty |
|---|---|---|---|---|
| No. | Ingredients | role | w/w | (kg) |
| 1 | Valerian Extract | Active | 32.000 | 0.320 |
| 2 | Hydroxypropyl | Carrier | 67.500 | 0.675 |
| | methylcellulose | | | |
| 3 | Colloidal Silicon Dioxide | Anticaking | 0.500 | 0.005 |
| | | agent | | |
| 4 | Ethanol | Solvent | Q.S. | Q.S. |
| 5 | Water | Solvent | Q.S. | Q.S. |
| | Total (Solid Content) | | 100.000 | 1.000 |

The valerian extract (0.320 kg) was weighed and added to the water under stirring to prepare a uniform dispersion. Hydroxypropyl methylcellulose (0.67 kg) was added to the purified water in separate vessel under stirring to prepare a uniform Solution. A Hydroxypropyl methylcellulose solution was added into valerian extract dispersion under stirring for a required period. In the next step spray dried the dispersion to obtain free flowing powder. The anticaking agent (0.005 kg) was added to spray dried powder.

### Example 2

| Sr. | | Functional | % | Qty |
|---|---|---|---|---|
| No. | Ingredients | role | w/w | (kg) |
| 1 | Valerian Extract | Active | 32.000 | 0.320 |
| 2 | Hydroxypropyl | Carrier | 67.500 | 0.675 |
| | methylcellulose | | | |
| 3 | Colloidal Silicon | Anticaking | 0.500 | 0.005 |
| | Dioxide | agent | | |
| 4 | Water | Solvent | Q.S. | Q.S. |
| | Total (Solid Content) | | 100.000 | 1.000 |

The process for the preparation of the valerian composition has been defined in stepwise manner as follows:

The Hydroxypropyl methylcellulose (0.675 kg) and valerian extract (0.320 kg) were weighed accurately and added to the purified water under stirring to prepare a uniform dispersion. Spray dried the dispersion to obtain free flowing powder. The anticaking agent (0.005 kg) was added to spray dried powder.

### Beadlets Formulation Preparation

### Example 3

The process for the preparation of the valerian composition in stepwise manner as follows:

| | | | | |
|---|---|---|---|---|
| 1 | Valerian Extract | Active | 32.000 | 0.320 |
| 2 | Hydroxypropyl methylcellulose | Binder | 2.000 | 0.020 |
| 3 | Microcrystalline cellulose | Carrier/Diluent | 64.000 | 0.640 |
| 4 | Colloidal silicon dioxide | Anticaking agent | 1.000 | 0.010 |
| 5 | Sunflower Lecithin | Bioenhancer | 1.000 | 0.010 |
| | (Phosphatidylcholine) | | | |
| 6 | Water | Solvent | Q.S. | Q.S. |
| 7 | Ethanol | Solvent | Q.S. | Q.S. |
| | Total (Solid Content) | | 100.000 | 1.000 |

Phosphatidylcholine (0.010 Kg), and valerian extract (0.320 Kg) were weighed and added to the purified water under stirring to prepare a uniform dispersion. Hydroxypropyl methylcellulose (0.020 Kg) was weighed and added to the purified water in separate vessel under stirring to prepare a solution. The obtained Hydroxypropyl methylcellulose solution was added into valerian extract dispersion to form a uniform dispersion. Microcrystalline cellulose (0.640 Kg) and Colloidal silicon dioxide (0.010 Kg) were weighed accurately and loaded in an RMG container and dry mixed at appropriate impeller speed to form a uniform blend. The obtained valerian extract dispersion was added gradually to the dry blend in RMG to obtain wet mass/wet granule. The wet mass was then passed through an extruder to obtain extrudes. Extrudes were loaded on a pitch spheronizer plate to obtain spherical beadlets/pellets. Spherical beadlets were loaded in a tray dryer for drying. Dried beadlets were sifted to obtain desired particle size distribution.

### Example 4

| Sr. | | Functional | % | |
|---|---|---|---|---|
| No. | Ingredients | Role | W/W | Qty.(Kg) |
| 1 | Valerian Extract | Active | 32.000 | 0.320 |
| 2 | Xanthan Gum | Binder | 1.000 | 0.010 |
| 3 | Microcrystalline cellulose | Carrier/Diluent | 64.000 | 0.640 |
| 4 | Colloidal silicon dioxide | Anticaking | 1.000 | 0.010 |
| | | agent | | |
| 5 | Sunflower Lecithin | Bioenhancer | 1.000 | 0.010 |
| | (Phosphatidylcholine) | | | |
| 6 | Polysorbate 80 | Emulsifier | 1.000 | 0.010 |
| 7 | Water | Solvent | Q.S. | Q.S. |
| | Total (Solid Content) | | 100.000 | 1.000 |

Phosphatidylcholine (0.010 Kg), and valerian extract (0.320 Kg) were weighed and added to the purified water under stirring to prepare a uniform dispersion. Xanthan Gum (0.010 Kg) was weighed and added to the purified water in separate vessel under stirring to prepare a solution. The obtained Xanthan gum solution was added into valerian extract dispersion to form a uniform dispersion. Microcrystalline cellulose (0.640 Kg) and Colloidal silicon dioxide (0.010 Kg) were weighed accurately and loaded in an RMG container and dry mixed at appropriate impeller speed to form a uniform blend. The obtained valerian extract dispersion was added gradually to the dry blend in RMG to obtain wet mass/wet granule. The wet mass was then passed through an extruder to obtain extrudes. Extrudes were loaded on a pitch spheronizer plate to obtain spherical beadlets/pellets. Spherical beadlets were loaded in a tray dryer for drying. Dried beadlets were sifted to obtain desired particle size distribution.

According to further embodiment of the present invention is provided valerian composition comprises valerian extract which improves and maintains the quality sleep, improves time to sleep, improves sleep duration, reduces sleep disturbances, and maintain healthy stress levels.

The present study is proposed to assess for efficacy of valerian composition comprising of valerian extract as defined in the present invention and use for prevention and improvement on sleep disorders.

### Study Objective

The main objective of study was to investigate the effects of Valerian composition comprising of Valerian extract as compared to melatonin on sleep and related behaviors in rodent models.

### Material and Methods

### Experimental Animals:

The study was designed to investigate the effects of valerian extract and melatonin on sleep and related behaviors in rodent model. Five male BALB/c mice per treatment arm (age: 8 weeks, weight: 20±3 g) were housed in a controlled environment with a 12:12-h light-dark cycle at 22° C. and provided with mice chow and water ad libitum. All experiments have been conducted under the National Institutes of Health's Guidelines for the Care and Use of Laboratory Animals and approved by the Ethics Committee.

The animals were further randomly allocated into 5 groups as defined below:
Group 1 (Control)—This group of animals were treated only with saline.
Group 2 (C)—This group of animals received caffeine (7.5 mg/kg) followed by saline
Group 3 (CM)—This group of animals received Caffeine followed by Melatonin (2 mg/kg) (CM)
Group 4 (CVA1)—This group of animals received Caffeine followed by Valerian composition comprising of Valerian extract (100 mg/kg)
Group 5 (CVAII)—This group of animals received Caffeine followed by Valerian composition comprising of Valerian extract (300 mg/kg)

### Groups for Sleep Parameters (Sleep Duration and Sleep Latency)

1) Control: Saline followed by saline
2) P: Pentobarbital followed by saline
3) PM: Pentobarbital followed by Melatonin of dose 2 mg/kg.
4) PVA I Pentobarbital followed by Valerian composition comprising of Valerian extract of dose 100 mg/kg.
5) PVA II: Pentobarbital followed by Valerian composition comprising of Valerian extract of dose 300 mg/kg.

### Experimental Design & Methodology:

### Treatment I:

According to experimental design and methodology groups were bifurcated the treatment into two groups namely treatment I and treatment II:
1) Monitoring the effect of caffeine and Valerian composition on brain activity
2) Monitoring the effect of Valerian composition on sleep parameters

In order to monitor the effects of caffeine and valerian composition on brain activity, EEG recording was performed. Mice was stabilized under a stereotaxic frame and an incision was made on the skin along the anterior-posterior line of the skull. Left cortex was removed gently with a dental drill. Two Ag-AgCl sphere electrodes were placed over the left hemisphere with the reference electrode attached on the pinna.

Working solutions of caffeine, melatonin, and valerian were prepared on a daily basis and administered intraperitoneally (i.p.). Based on the previous studies caffeine-induced insomnia is dose-dependent with 7.5 mg/kg i.p. caffeine maintaining wakefulness for at least 2 h. Electrical activity of the brain was monitored for 90 minutes following the caffeine injection and spike frequency/spike amplitude analysis was performed via the Labchart 7.3.3 program. Ninety (90) minutes after the caffeine injection, animals were deeply anesthetized with 4% isofluorane (30% O₂, remainder N₂O). Brains were rapidly removed, frozen on dry ice and stored at −80° C. for further analysis.

### A. Biochemical and Oxidative Stress Markers

Serum melatonin, serotonin, and dopamine was measured by ELISA kits. Brain levels of MDA was measured by HPLC. Brain activities of SOD, CAT and GPHx was measured using ELISA kits.

### B. Protein Analyses

Protein concentrations were determined using Western blot analysis. 50 μg of protein was electrophoresed on 4-15% Tris-Glycine polyacrylamide gels and transferred to Immobilon-P PVDF membranes, blocked for 1 h in 5% skim milk and incubated overnight at 4° C. with either GABA_{A}R2, GABA_{B} R1, GABA_{B}-R2, 5H-T1A, GluA1, GluN1, GluN2 A, Bcl-2, Bax, and Caspase-3. Membranes were then incubated with horseradish peroxidase (HRP) conjugated IgG secondary antibody. Bands were quantified using Image software and normalized to actin as a loading control.

### Treatment II

The groups were administered valerian composition and then, 45 min later, pentobarbital (a hypnotic dose, 42 mg/kg) injected into the left side of the abdomen. After injection, the mice were placed in individual cages and subjected to measurements of sleep latency and duration. Sleep latency defined as the period between pentobarbital injection and sleep onset, and sleep duration denotes the time elapsed between the righting reflex loss and recovery. Mice that failed to fall asleep within 10 min after pentobarbital injection were excluded from the experiments.

### Data Analysis Method

Data was given as mean±SEM. Sample size was calculated based on a power of 85% and a p-value of 0.05. Given that assumption, a sample size of five per treatment was calculated and the data was analyzed using the GLM procedure of SAS (SAS Institute: SAS User's Guide: Statistics). The treatments were compared using ANOVA and Student's unpaired t-test; P<0.05 was considered statistically significant.

FIG. 1 shows the effect of melatonin and valerian extracts (VA) on brain electrical activity. Spike frequency and amplitude analyses on ECoG recording. Values are represented as mean S.E.M. for each group, n=5. A) Spike amplitude (%) and B) Spike frequency (%).

### Conclusion:

We first analyzed the EcoG recording to evaluate electrical brain waves, spike frequency and spike amplitude. The spike frequencies were the highest, but amplitudes were the lowest throughout the recording in the Caffeine (C) group.

Spike amplitudes (%) were higher at all time points for 300 mg/kg (CVA2) group when compared to all the groups. Further, 100 mg/kg (CVA1) also showed higher amplitude at 15-30, 30-45, 45-60 and 60-75 min of interval as compared to C group. CVA2 performed better than CM group at all time points.

Spike frequency was recorded to be lower in both 100 mg/kg (CVA1) and 300 mg/kg (CVA2) groups compared to C group. Further both CVA1 and CVA2 performed almost similar to CM group.

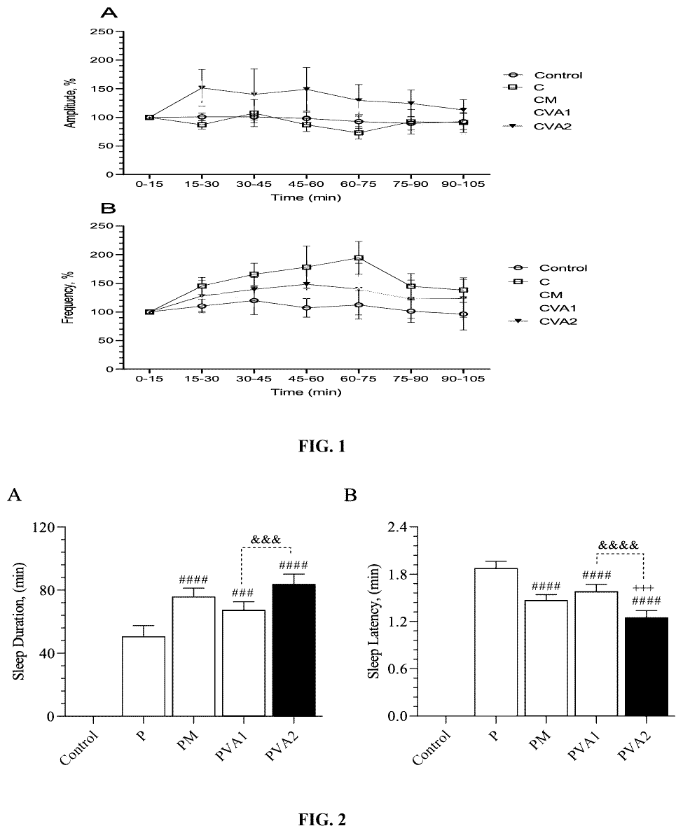

FIG. 2 shows the effects of melatonin and valerian extracts (VA) on the sleep duration (A) and sleep latency (B) after pentobarbital administration. Each bar represents the mean and standard error of the mean. Tukey's post-hoc test were used for comparing the results among different treatment groups, and statistical significance between groups is shown by: ###P<0.001; ####P<0.0001 compared to P group, +P<0.05 compared to PM group, and &&&P<0.001; &&&&P<0.0001 compared to PVA I group.

### Conclusion:

Both PVA1 and PVA2 groups showed significant increase in sleep duration and significant decrease in sleep latency as compared to P group. PVA2 was significantly better than PVA1 on both sleep duration and sleep latency measures. Further, no statistical difference was observed between PM and PVA1 and PM and PVA2 groups. It can be concluded that both PVA1 & PVA2 have positive impact on sleep.

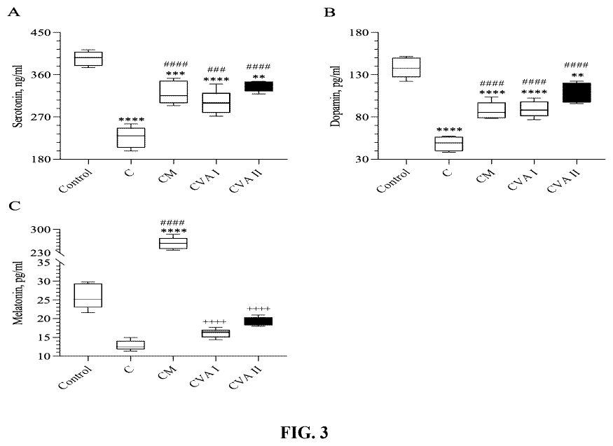

FIG. 3 shows the effects of melatonin and valerian extracts (VA) on the serum of serotonin, dopamine, and melatonin. ANOVA and Tukey's post-hoc test were used for comparing the results among different treatment groups, and statistical significance between groups is shown by: ** P<0.01; *** P<0.001, **** P<0.0001 compared to control group, ###P<0.001; ####P<0.0001 compared to C group, ++++P<0.0001 compared to CM group.

### Conclusion:

CVA1, CVA2 and CM groups showed significant increase in serum serotonin and dopamine levels as compared to C group. No significant difference was seen between CM-CVA1 or CM-CVA2 groups on serum serotonin and dopamine levels.

Further, CM group reported significantly higher melatonin levels in serum as compared to all the other groups. Additionally, both CVA1 and CVA2 groups did not show significant difference in comparison to C group.

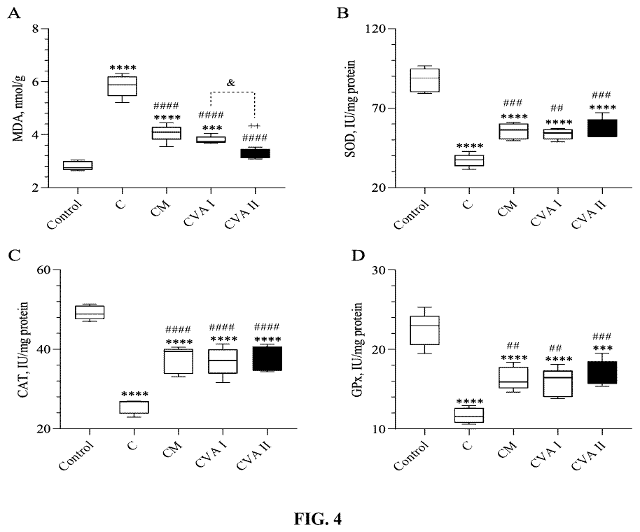

FIG. 4 shows the effects of melatonin and valerian extracts (VA) on the brain tissue MDA (A), SOD (B), CAT (C), and GPx. ANOVA and Tukey's post-hoc test were used for comparing the results among different treatment groups, and statistical significance between groups is shown by: *** P<0.001, **** P<0.0001 compared to control group, ##P<0.01; ###P<0.001; ####P<0.0001 compared to C group, ++P<0.01 compared to CM group, & P<0.05 compared to CVA I group.

### Conclusion:

CVA1, CVA2 and CM groups showed significant increase in antioxidant enzymes like SOD, CAT and GPx as compared to C group. No significant difference was seen between CM-CVA1 or CM-CVA2 groups on SOD, CAT and GPx.

Further, CVA1, CVA2 and CM groups recorded significant reduction in MDA levels as compared to C group. Additionally, CVA2 performed significantly better than CM and CVA1 groups. CVA1 did not show significant difference in comparison to CM group. It can be concluded that both CVA1 & CVA2 reduced oxidative stress.

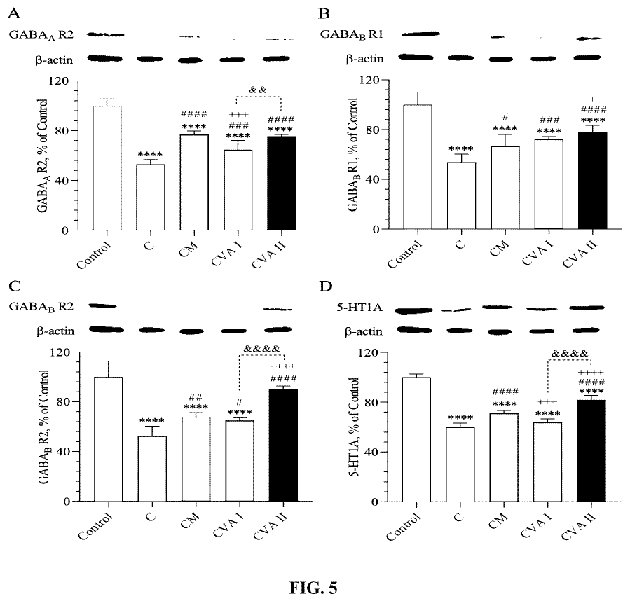

FIG. 5 shows the effects of melatonin and valerian extracts (VA) on the brain tissue GABAergic receptors GABA_{A}R2 (A), GABA_{B} R1 (B), GABA_{B} R2 (C), and serotonergic receptor 5-HT1A (D). ANOVA and Tukey's post-hoc test were used for comparing the results among different treatment groups, and statistical significance between groups is shown by: **** P<0.0001 compared to control group, #P<0.05; ##P<0.01; ###P<0.001; ####P<0.0001 compared to C group, +P<0.05; +++P<0.001; ++++P<0.0001 compared to CM group, and && P<0.01; &&&&P<0.0001 compared to CVA I group.

### Conclusion:

CVA1, CVA2 and CM groups showed significant increase in GABA_{A} R2, GABA_{B} R1 and GABA_{B} R2 as compared to C group. Further, CVA2 performed significantly better than CVA1 in increasing the levels of GABA_{A} R2 and GABA_{B} R2. Additionally, GABA_{A} R2 was significantly lower in CVA1 group compared to CM group. Both GABA_{B} R1 and GABA_{B} R2 were significantly increased in CVA2 group compared to CM group.

The 5-HT1A levels were significantly increased in CM and CVA2 groups compared to C group. CVA2 showed significantly higher levels of 5-HT1A than CVA1. CVA2 performed significantly better than CM group whereas CVA1 group was significantly lower than CM group on 5-HT1A expression.

It can be concluded that valerian extract enhanced the levels of inhibitory receptors which were declined by caffeine induction.

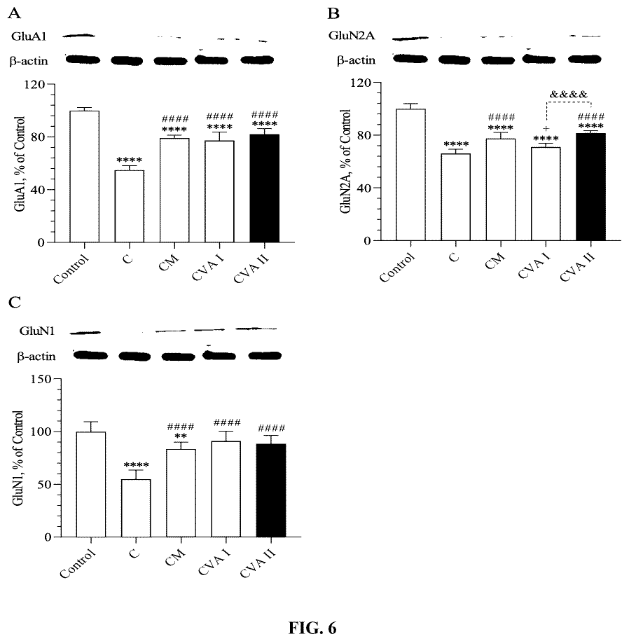

FIG. 6 shows the effects of melatonin and valerian extracts (VA) on the brain GluA1(A), GluN2A (B), and GluN1 (C). ANOVA and Tukey's post-hoc test were used for comparing the results among different treatment groups, and statistical significance between groups is shown by: **P 0.01; **** P<0.0001 compared to control group, ####P<0.0001 compared to C group, +P<0.05 compared to CM group, and &&&&P<0.0001 compared to CVA I group.

### Conclusion:

CVA1, CVA2 and CM groups showed significant increase in GluA1 and GluN1 receptors as compared to C group. Additionally, CVA2 and CM groups only showed significant increase in GluN2A receptors as compared to C group.

No significant difference was seen between CM, CVA1 and CVA2 groups for the expression of GluA1 and GluN1 receptors.

Expression of GluN2A receptors was significantly lower in CVA1 group as compared to CM group whereas CVA2 showed significantly higher levels of GluN2A receptors as compared to CVA1.

It can be concluded that Valerian extract and melatonin both increase the Glutamate receptors after caffeine-induced deprivation.

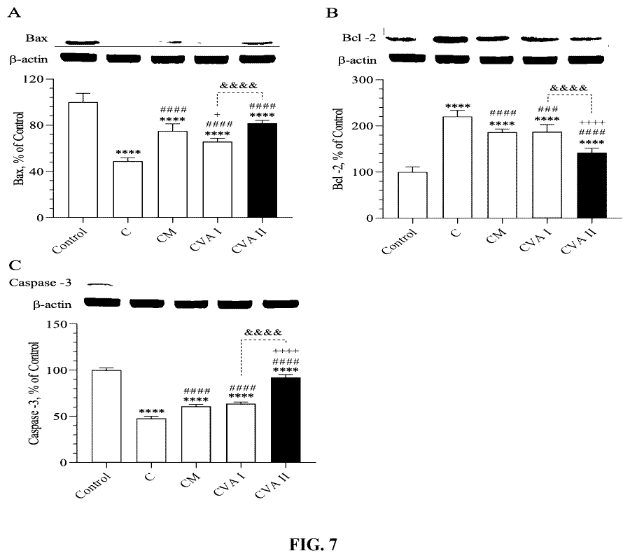

FIG. 7 shows the effects of melatonin and valerian extracts (VA) on the brain Bax(A), Bcl-2 (B), and Caspase-3 (C). ANOVA and Tukey's post-hoc test were used for comparing the results among different treatment groups, and statistical significance between groups is shown by: **** P<0.0001 compared to control group, ###P<0.001; ####P<0.0001 compared to C group, +P<0.05; ++++P<0.0001 compared to CM group, and &&&& P<0.0001 compared to CVA I group.

### Conclusion:

CVA1, CVA2 and CM groups showed significant increase in Bax and Caspase-3 and significant decrease in Bcl-2 as compared to C group.

CVA1 showed significantly lower levels of Bax expression compared to CM group whereas no significant difference was seen between CVA2 and CM groups. Further, CVA2 showed significantly higher levels of Bax expression compared to CVA1.

CVA2 showed significantly higher levels of Caspase-3 expression compared to both CVA1 and CM groups whereas no significant difference was seen between CVA1 and CM groups.

CVA2 showed significantly lower levels of Bcl-2 expression compared to both CVA1 and CM groups whereas no significant difference was seen between CVA1 and CM groups. From this it can be concluded that CVA2 showed more neuroprotective activity than all the other groups.

### Abbreviations:

ELISA: Enzyme-linked immunoassay
MDA: Malondialdehyde
SOD: Superoxide dismutase
CAT: Catalase
GPHx: Glutathione peroxidase
GABA_{A}R2: Gamma-aminobutyric acid type A receptor subunit 2
GABA_{B} R1: Gamma-aminobutyric acid type B receptor subunit 1
GABA_{B}-R2: Gamma-aminobutyric acid type B receptor subunit 2
5HT1A: Serotonin 1A receptor
GluA1: Glutamate receptor 1
GluN1: Glutamate [NMDA] receptor subunit zeta-1
GluN2 A: Glutamate [NMDA] receptor subunit zeta-1
Bcl-2: B-cell lymphoma 2
Bax: Apoptosis regulator
Caspase-3: Cysteine-dependent aspartate-directed proteases-3

We claim:

1. A composition comprising Valerian officinalis L extract wherein the valerenic acids are in range of 5% w/w to 20% w/w of the extract.

2. A composition comprising:
i) Valerian extract in an amount ranging from 20% to 33% w/w of the composition;
ii) at least one hydrophilic carrier or diluent in an amount ranging from of 30% to 67% w/w of the composition; and
iii) anticaking agent in an amount ranging from 0.01% to 1% w/w of the composition.

3. The composition according to claim 1, wherein the composition further comprises a pharmaceutically or nutraceutically acceptable excipient selected from the group consisting of a hydrophilic carrier, a diluent, an anticaking agent, a solvent and a mixture thereof.

4. The composition according to claim 3, wherein the hydrophilic carrier or diluent is selected from the group consisting of hydroxypropyl methyl cellulose and cellulose derivatives, microcrystalline cellulose, polyacrylates, polyethylene glycols, povidones, starch and starch derivatives, chitosan, gums, polyvinyl alcohol, dextran, polylactic-co-glycolic acid, polyethylene glycol, maltodextrin and/or a combination thereof.

5. The composition according to claim 3, wherein the anticaking agent is selected from the group consisting of silicon dioxide, dicalcium phosphate, magnesium stearate, stearic acid, mannitol, and combination thereof.

6. The composition according to claim 3, wherein the solvent used is selected from water, methanol, ethanol, Iso propyl alcohol, acetone, ethyl acetate, methylene dichloride, and/or a combination thereof.

7. The composition according to claim 1, the composition comprising:
i) valerian extract in an amount ranging from 32% w/w of the composition,
ii) hydroxypropyl methylcellulose in an amount of 67.5% w/w of the composition, and
iii) colloidal silicon dioxide (Aerosol) in an amount of 0.5% w/w of the composition.

8. A method for preventing and/or improving sleep disorders in a subject, comprising administering the composition according to claim 1 to a subject in need thereof.

9. A method for improving quality of sleep, improving time to sleep, improving sleep duration, reducing sleep disturbances, and maintaining healthy stress levels in a subject, the method comprising administering the composition according to claim 1 to a subject in need thereof.

10. The method according to claim 9, wherein the subject is human and/or animal.

11. The composition according to claim 1, wherein the composition is in a dose form comprising 50 mg/kg to 400 mg/kg of body weight of a subject.

12. A process for the preparation of the composition according to claim 1, the process comprising:
a) preparing a solution of valerian extract in water to form dispersion,
b) preparing a solution of binder,
c) adding a solution obtained in step b to the solution obtained in step a followed by stirring to obtained homogeneous solution,
d) spray drying solution obtained in step c to obtain free flowing powder, and
e) adding an anticaking agent in powder obtained from step d to produce the composition according to claim 1.

13. A Valerian officinalis L composition comprising valerian extract, wherein the valerian extract comprises:
(i) valerenic acid in an amount ranging from 1% w/w to 20% w/w of the valerian extract,
(ii) hydroxy valerenic acid in an amount ranging from 0% w/w to 5% w/w of the valerian extract, and
(iii) acetoxy valerenic acid in an amount ranging from 0% w/w to 10% w/w of the valerian extract.
